(19) United States
(12) Patent Application Publication
(10) Pub. No.: US 2026/0260712 A1
(43) Pub. Date: Sep. 3, 2026

(54) PROCESSES, MACHINES, AND ARTICLES OF MANUFACTURE RELATED TO PREDICTING DOSE RESPONSE OF COMPOUNDS

(71) Applicant: Flagship Pioneering Innovations VI, LLC, Cambridge, MA (US)

(72) Inventors: Nathan Edward Sanders, North Andover, MA (US); Hok Hei Tam, Newton, MA (US); Changchang Liu, Cambridge, MA (US); Xinmeng Li, Cambridge, MA (US)

(21) Appl. No.: 18/993,335

(22) PCT Filed: Jul. 11, 2023
(86) PCT No.: PCT/US2023/069974

§ 371 Date: Jan. 10, 2025

Related U.S. Application Data
(60) Provisional application No. 63/368,161, filed on Jul. 12, 2022.
(60) Provisional application No. 63/375,730, filed on Sep. 15, 2022.

(51) Int. Cl. G16C 20/30; G16C 20/70

(52) U.S. Cl. CPC G16C 20/30; G16C 20/70

(57) ABSTRACT

A computational model is constructed to output, and allow supervision based on, a set of values that describes the item quantity dependent values. Such a model can predict the dose-response of a compound with respect to some bioactivity. The output of the model can be an array of values sampling the dose-response curve or set of parameters of a function that models the dose-response curve. Known values for researched items can be used as supervisory information during training. A dynamic loss function dynamically weights two loss function components: one that optimizes the model for classification, and another that optimizes the model for regression, or curve fitting.

### BACKGROUND

Machine learning generally involves using data about one set of items for which a property is known, such as classifications for the items, to train a computational model that in turn can make predictions about what that property should be for other items, for which that property is not known. While there is a wide range of possible applications of this general concept of machine learning, practical applications can be hard to implement for many reasons.

### SUMMARY

This Summary introduces a selection of concepts in simplified form that are described further below in the Detailed Description. This Summary neither identifies key or essential features, nor limits the scope, of the claimed subject matter.

Machine learning techniques can be used to build a computer system that can predict values which are dependent on an item quantity, such as dose-dependent bioactivity of a compound, a dose-response curve, or other relationship between a concentration of a compound and its effect. To do so, the computer system has access to data representing a set of researched items for which item quantity dependent information is available. The computer system also has access to data representing potential candidate items. For each potential candidate item, respective information is not known about item quantity dependent values. The computer system applies machine learning techniques to train a computational model using the data representing the researched items and their known properties. The computer system applies the trained computational model to the data representing the potential candidate items. In response, the trained computational model outputs one or more predicted values which depend on item quantities of the potential candidate items.

To train such a computational model, a selected subset of the researched items is used, called a training set. Researched items in the selected subset have information characterizing values which depend on a quantity of the researched item, such as known dose and response information. The selected researched items can be those for which some information about the dose-response curve is known.

After training, the trained computational model is applied to a selected subset of potential candidate items, called a target set, to generate a respective result set. The result set comprises data representative of a set of predicted candidate items from among the plurality of potential candidate items. The trained computational model predicts, for a potential candidate item, one or more respective values which are dependent on quantities of the potential candidate item, such as a predicted dose-response curve for the item. The predicted candidate items are those potential candidate items for which the trained computational model generates a representation of the item quantity dependent values. Within the predicted candidate items, there may be some that can be identified as “active”, and others that can be identified as “inactive”. An “active” item has item quantity dependent values which indicate a significant dependence of the value on the item quantity, such as a compound having a dose-response curve indicating that the compound is active, i.e., the compound is predicted to generate a significant response given a certain dose of that compound. For an “inactive” item, the value for the item typically has little, if any, variation in response to a change in the item quantity, such as an inactive compound which is not predicted to generate any significant response to a dose of that compound.

Machine learning techniques can be challenging to apply to make predictions of values which are dependent on quantities of items for several reasons, of which some are the following.

Within a training set, it is generally considered good practice to have both positive and negative examples of items with respect to some characteristics. In the example of dose-response curves of compounds and related bioactivity, it is desirable to have, on the one hand, some researched items for which there is no significant bioactivity, and, on the other hand, some researched items for which there is known significant bioactivity and for which the dose-dependence of that bioactivity also is known at least in part. However, building a model that predicts both whether a compound has bioactivity and the item quantity-dependency of that bioactivity can challenge conventional machine learning techniques, because such predictions involve both classification problems and regression problems.

Also, in machine learning, a training set is selected from a set of researched items for which quantifiable information about the item quantity dependent values is known. Because such data generally is obtained from a set of physical experiments, this “known” information may be, and typically is, incomplete.

In addition, such experiments typically are performed in replicates to capture experimental variation. This experimental variation can be substantial. Variation in measurements also may arise through performing different experiments, or performing experiments with different equipment, source material, or personnel. For example, some laboratory environments are more well controlled and well equipped than others to produce repeatable and reliable measurements. As another example, the nature of a property of an item and how it can be measured can introduce uncertainty in the information available about properties of researched items. Such variation in data in a training set can adversely impact the performance of a trained computational model.

To address such problems, a variety of machine learning techniques can be used.

A computational model is constructed to output, and allow supervision based on, a set of values that describes the item quantity dependent values. For the dose-response of a compound with respect to some bioactivity, this output can be an array of values sampling the dose-response curve or set of parameters of a function that models the dose-response curve. With such a model, the known values for researched items can be used as supervisory information during training.

In the field of bioactivity of compounds, many dose-responses curves are described by nonlinear sigmoid models, such as a Hill function. For an individual item, statistics about the quantity-effect relationship, such as potency, efficacy, and slope, can be computed based on one or more inferred Hill functions for the item. In other fields and with other kinds of items, the item quantity dependent values may be understood using other parametric models.

One or more values from such parametric models is indicative of whether the value for the item is quantity dependent. For example, a value from a parametric model may indicate a maximum or asymptotic value, or an average value, or other value which is substantially different from zero or other baseline value. In the field of bioactivity of compounds, a maximum value of a Hill function representing a dose-response curve can indicate whether a compound may achieve a given level of bioactivity.

To address the problem that the model must perform both classification and regression, such that it classifies an item to have significant activity (or not) and also predicts its item quantity dependent values, a dynamic loss function is used. In particular, the loss function is a dynamically weighted combination of two loss function components. The first loss function component optimizes the model for classification; the second loss function component optimizes the model for regression, or curve fitting. In one implementation, the loss function is initially heavily weighted in favor of the classification component. This initial weighting reduces the likelihood that the model attempts to fit the data for an item to a curve where that data instead indicates that the item does not have quantity dependent values. For example, if a compound does not have a certain level of maximum bioactivity, then the model should not attempt to map that compound's data to a dose-response curve for that bioactivity. As training epochs progress, the weighting towards the regression-based loss function increases.

In some implementations, the training set includes researched items for which the respective quantitative information describing item quantity dependent values is incomplete. Also, in such implementations using incomplete information, these techniques enable sparse dose response curves (e.g., with as few as one or two data points) to be used to supervise a dose response regression model. With these techniques, such sparse data is still suitable for training because meaningful information can be extracted by the model. In some implementations with incomplete information, given the data that is known and the parametric model that represents the dose-response curve, the known quantitative information can be processed, for example using interpolation, to complete the set of quantity dependent values for that item.

In some implementations, where the supervising data include replicates, the computational model is constructed to include parameters representing uncertainty associated with the measurements of the item quantity dependent values. In some implementations, such uncertainty parameters also can be trained, so that the trained computational model outputs a set of values for these parameters associated with any prediction.

In some implementations, the computational model can be an ensemble of two models: one model implementing a regression model which is trained to predict a curve representing item quantity dependence of a value, and another model implementing a classification model which is trained to predict whether an item is active. In some implementations of this technique, during training, the classification model can be trained, using a training set of active and inactive items, to predict whether items are active or not. The regression model is trained using the active items to predict item quantity dependence parameters. During inference, the classification model is applied first to determine whether an item is predicted to be active or inactive. In some implementations, particularly if the training set has extreme class imbalance (i.e., there are more inactive items than active items), the regression model can be applied only to the subset of items predicted to be active by the classifier. In such cases, regression models often perform poorly because a dominating set of compounds offer essentially no signal for regression (inactive items have zero activity at all doses) while only a small subpopulation provides a useful signal. By using the classification loss, the regression model can be trained to better distinguish between active and inactive compounds and model performance can be improved.

These machine learning techniques help a computer system to make predictions about other items that may have item quantity dependent values, such as dose-response curves for compounds where information is limited.

Accordingly, in one aspect, a computer system predicts item quantity dependent properties of items. For example, a computer system can predict an item quantity dependent property of items based on quantitative information obtained from measurements of the property of items. In one aspect a computer system predicts dose response of compounds. In one aspect a computer system predicts bioactivity and dose response of compounds.

Accordingly, in one aspect, computer system for predicting an item quantity dependent property of items based on quantitative information obtained from measurements of the property of items includes a processing system comprising a processing device and computer storage storing computer program instructions. When processed by the processing device, the computer program instructions configure the processing system to train a computational model using data representing researched items. The data representing researched items includes, for each researched item, respective quantitative information describing item quantity dependent values corresponding to different quantities of the researched item. The computer system inputs data representing a plurality of potential candidate items to the trained computational model such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer-implemented method for predicting an item quantity dependent property of items based on quantitative information obtained from measurements of the property of items includes training a computational model using data representing researched items. The data representing researched items includes, for each researched item, respective quantitative information describing item quantity dependent values corresponding to different quantities of the researched item. The method further includes inputting data representing a plurality of potential candidate items to the trained computational model such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer program product for predicting an item quantity dependent property of items based on quantitative information obtained from measurements of the property of items includes computer storage storing computer program instructions. When processed by a processing device of a computer, the computer program instructions configure the computer to train a computational model using data representing researched items. The data representing researched items includes, for each researched item, respective quantitative information describing item quantity dependent values corresponding to different quantities of the researched item. The computer further inputs data representing a plurality of potential candidate items to the trained computational model such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer system for predicting an item quantity dependent property of items based on quantitative information obtained from measurements of the property of items includes a processing system comprising a processing device and computer storage storing computer program instructions. When processed by the processing device, the computer program instructions configure the processing system to instantiate a trained computational model. The trained computational model has been trained using data representing researched items including, for each researched item, respective quantitative information describing item quantity dependent values corresponding to different quantities of the researched item. The processing system further inputs data representing a plurality of potential candidate items to the trained computational model such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer-implemented method for predicting an item quantity dependent property of items based on quantitative information obtained from measurements of the property of items includes instantiating, in a computer system, a trained computational model. The trained computational model has been trained using data representing researched items including, for each researched item, respective quantitative information describing item quantity dependent values corresponding to different quantities of the researched item. The method further includes inputting data representing a plurality of potential candidate items to the trained computational model, such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer program product for predicting an item quantity dependent property of items based on quantitative information obtained from measurements of the property of items includes computer storage storing computer program instructions. When processed by a processing device of a computer, the computer program instructions configure the computer to instantiate a trained computational model. The trained computational model has been trained using data representing researched items including, for each researched item, respective quantitative information describing item quantity dependent values corresponding to different quantities of the researched item. The computer further inputs data representing a plurality of potential candidate items to the trained computational model such that the trained computational model outputs a result set. The result set includes respective predicted information, for each predicted candidate item from among the plurality of potential candidate items, describing item quantity dependent values for the predicted candidate item for a plurality of different quantities. In some implementations the result set is stored in computer storage, such as a database.

In one aspect, a computer system for predicting an item quantity dependent property of items based on quantitative information obtained from measurements of the property of items includes a processing system comprising a processing device and computer storage storing computer program instructions. When processed by the processing device, the computer program instructions configure the processing system to instantiate a computational model. The processing system applies features representing researched items to inputs of the computational model. The data representing researched items includes, for each researched item, respective quantitative information describing item quantity dependent values corresponding to different quantities of the researched item. In response to features describing a researched item, outputs of the computational model describe respective predicted item quantity dependent values for the researched item. The processing system updates parameters of the computational model based on comparisons of the outputs of the computational model to the data representing researched items.

In one aspect, a computer-implemented method for predicting an item quantity dependent property of items based on quantitative information obtained from measurements of the property of items includes instantiating in a computer system a computational model. The computer system applies features representing researched items to inputs of the computational model. The data representing researched items includes, for each researched item, respective quantitative information describing item quantity dependent values corresponding to different quantities of the researched item. In response to features describing a researched item, outputs of the computational model describe respective predicted item quantity dependent values for the researched item. The method further includes updating parameters of the computational model based on comparisons of the outputs of the computational model to the data representing researched items.

In one aspect, a computer program product for predicting an item quantity dependent property of items based on quantitative information obtained from measurements of the property of items includes computer storage storing computer program instructions. When processed by a processing device of a computer, the computer program instructions configures the computer to instantiate a computational model. The computer further applies features representing researched items to inputs of the computational model. The data representing researched items includes, for each researched item, respective quantitative information describing item quantity dependent values corresponding to different quantities of the researched item. In response to features describing a researched item, outputs of the computational model describe respective predicted item quantity dependent values for the researched item. The computer further updates parameters of the computational model based on comparisons of the outputs of the computational model to the data representing researched items.

In any of the foregoing, one or more of the following features can be included. The respective predicted information for each predicted candidate item includes a respective set of values for a set of quantities of the item describing a sigmoidal curve. The respective quantitative information for each researched item comprises a respective set of values for a set of quantities of the item describing a sigmoidal curve. The respective quantitative information describing item quantity dependent values corresponding to different quantities of the researched item comprises sparse data for at least some of the researched items. The sparse data includes single dose high throughput screening data for a plurality of researched items. The respective quantitative information describing item quantity dependent values corresponding to different quantities of the researched item includes multiple dose data for at least some of the researched items.

In any of the foregoing, one or more of the following features can be included. The computational model comprises a set of inputs receiving features representing items, a latent parameter model implementing a function mapping the features to outputs providing parameters representing a curve, wherein the curve represents how values for items depend on quantity of items in a class of items. The computational model further comprises a conversion module having inputs connected to receive the parameters representing the curve, a conversion model implementing a function mapping the parameters representing the curve to outputs providing data representing an array of item quantity dependent values corresponding to the curve.

In any of the foregoing, one or more of the following features can be included. To train the computational model, supervisory information includes the quantitative information describing item quantity dependent values corresponding to different quantities of the researched item. To train the computational model, the processing system is configured to compare the supervisory information and the output of the conversion module to generate updates to parameters of the computational model according to a loss function. The parameters of the computational model include parameters of the latent parameter model. The parameters of the computational model include parameters of the conversion model. The loss function comprises a dynamic loss function. The dynamic loss function comprises a weighted combination of a first loss function and a second loss function. The first loss function comprises a classification-based loss function. The second loss function comprises a regression-based loss function. The dynamic loss function applies a weight that varies across training epochs during training.

In any of the foregoing, one or more of the following features can be included. The items are compounds and the item quantity dependent values are a dose-response curve. The dose-response curve represents bioactivity of the compound.

In any of the foregoing aspects, the researched compounds can include one or more of small synthetic molecules or drugs.

In any of the foregoing aspects, the potential candidate compounds can include one or more of proteins found in food, compounds found in food, compounds that are generally recognized as safe for human consumption, large naturally occurring molecules, or other molecules with a history of human exposure.

In any of the foregoing aspects, the quantitative information describing item quantity dependent values corresponding to different quantities of the researched item can include measured and quantified bioactivity related to a protein in response to presence of the compound in a living thing.

In any of the foregoing aspects, the bioactivity can include bioactivity related to a selected protein in response to presence of a compound in a living thing. The selected subset of the plurality of researched compounds used for training can include researched compounds having information characterizing bioactivity related to the selected protein.

In any of the foregoing aspects, bioactivity related to a protein can include bioactivity related to a concentration of the protein present in a living thing.

In any of the foregoing aspects, bioactivity can include bioactivity related to a health condition of a living thing, or a phenotypic response of a living thing, such as a concentration of protein present in the living thing.

In any of the foregoing aspects, the living thing can include one or more of plants, mammals, animals, or humans.

In any of the foregoing aspects, the quantitative information describing item quantity dependent values corresponding to different quantities of the researched item can include a measured concentration of a protein in response to presence of a measured amount of a compound. The information can include an amount in a continuous or semi-continuous range indicating a concentration of an item in a sample. The information can include a concentration of another item related to the amount of protein present in a sample.

In any of the foregoing aspects, the computational model can be designed to predict whether the candidate compounds interact directly or indirectly with, or independently of, the respective selected protein. The interaction can be positive or negative. a computational model can be designed to predict whether the candidate compounds interact, when present with another compound, with the respective selected protein.

In any of the foregoing aspects, querying can include identifying one or more of: compounds that interfere with activity of a drug, foods containing compounds that interfere with activity of a drug, compounds that enhance activity of a drug, foods containing compounds that enhance activity of a drug.

In any of the foregoing aspects, querying can include aggregating interaction information for a plurality of compounds to characterize an overall effect of the plurality of compounds with respect to a health condition, or with respect to a drug.

The following Detailed Description references the accompanying drawings which form a part of this application, and which show, by way of illustration, specific example implementations. Other implementations may be made without departing from the scope of the disclosure.

### BRIEF DESCRIPTION OF THE DRAWINGS

FIG. 1 is a data flow diagram of an example implementation of a computer system that uses machine learning techniques to predict item quantity dependent values.

FIG. 2 is an example implementation of a data structure for data representing researched items, potential candidate items, and predicted candidate items.

FIG. 3A is an illustration of data structure representing experimental data for an item quantity dependent value.

FIG. 3B is an illustration of an example curve of an example parametric model that represents an item quantity dependent value.

FIG. 4 is a data flow diagram of an example implementation of a machine learning model that predicts parameters representing an item quantity dependent value.

FIG. 5 is a flow chart of an example operation using the machine learning model of FIG. 4.

FIG. 6 is a data flow diagram of an example implementation of a computer system that incorporates a dose-response model.

FIGS. 7 and 8 describe modeling uncertainty.

FIG. 9 describes applying prior information.

FIG. 10 is a block diagram of an example general purpose computer.

In the drawings, in the data flow diagrams, a parallelogram indicates an object that is an input to a system that manipulates the object or an output of such a system, whereas a rectangle indicates the system that manipulates that object.

### DETAILED DESCRIPTION

Machine learning techniques can be used to build a computer system that predicts properties of items. To do so, the computer system has access to data representing a set of researched items for which a property is known. The property which a researched item has is one from among a plurality of types of properties. The computer system also has access to data representing potential candidate items. For each potential candidate item, respective information is not known for at least one property among the plurality of types of properties. The computer system applies machine learning techniques to train a computational model using the data representing the researched items and their known properties, for a plurality of types of properties. The computer system applies the trained computational model to the data representing the potential candidate items. In response, the trained computational model outputs one or more predictions about whether the potential candidate items are likely to have a property from among the plurality of types of properties that the computational model is trained to predict.

The “potential candidate items” that are predicted by the computational model to have a property become “predicted candidate items” for that property. Laboratory or other experiments can be performed to verify the predictions output by the trained computational model. Verification transforms a prediction about a predicted candidate item into quantitative information about a known property of a researched item. Thus, an item can be a potential candidate item with respect to one type of property that is not known for the item, yet can also be a researched item with respect to another different type of property that is known for the item, and yet can also be a predicted candidate item with respect to yet another different type of property.

The property predicted by the trained computational model for the predicted candidate items using this technique is the predicted representation of the item quantity dependent values for that item. Within the predicted candidate items, there may be some that can be identified as “active”, and others that can be identified as “inactive”. An “active” item has item quantity dependent values which indicate a significant dependence of the value on the item quantity, such as a compound having a dose-response curve indicating that the compound is active, i.e., the compound is predicted to generate a significant response given a certain dose of that compound. For an “inactive” item, the value for the item typically has little, if any, variation in response to a change in the item quantity, such as an inactive compound which is not predicted to generate any significant response to a dose of that compound.

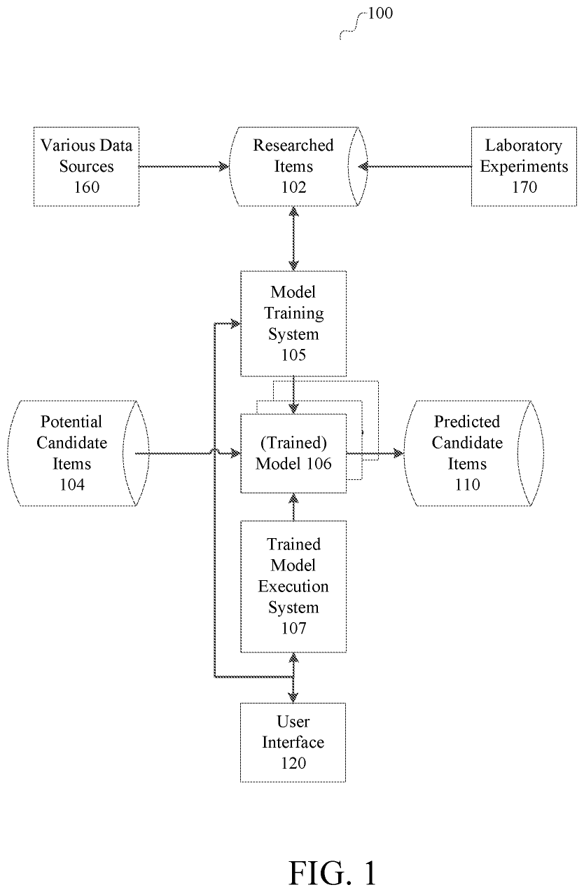

Referring to FIG. 1, an example implementation of a computer system that uses machine learning techniques to predict item quantity dependent values will now be described.

The computer system 100 has access to data 102 representing a set of researched items. A researched item is an item for which a property is known. The property which a researched item has is one from among a plurality of types of properties. The computer system 100 also has access to data 104 representing potential candidate items. A potential candidate item is an item for which the property is not known. Information about researched compounds can come from a various data sources 160, examples of which are described in more detail below, or from laboratory experiments 170, or both.

The computer system applies machine learning techniques, implemented by a model training system 105, to train a computational model 106 using data 102 representing the researched items and their known properties. The computer system, using the trained model execution system 107, applies the trained computational model 106 to data 104 representing potential candidate items. In response, the trained computational model outputs data 110 representing one or more predictions about whether the potential candidate items are likely to exhibit a property. Those potential candidate items that are predicted to have a property are predicted candidate items for that property.

One or more user interfaces 120 can be provided to enable a user to access the computer system 100. For example, a user interface can be provided to identify researched items for training sets for use by model training system 105. A user interface can be provided to identify potential candidate items to input to a trained model 106 to obtain predictions. A user interface can be provided to search the information about predicted candidate items 100 to identify items to be submitted for laboratory experiments (170).

Laboratory or other experiments can be performed to verify the predictions output by the trained computational model. The computer system 100 can have an input interface (not shown) through which data can be received that includes information characterizing verified properties based on laboratory or other experiments 170 performed to verify predictions. Through this interface, such data can be stored in the database 102 of researched items, thus transforming a prediction into quantitative information about a property of a researched item.

Items can include any of a variety of physical items, which may include machines, articles of manufacture, or compositions of matter, or any combination of these. Such physical items include compounds, and combinations of compounds, including various forms of such combinations (e.g., mixtures, solutions, alloys, conglomerates) or structure of such combinations (e.g., mechanical, electrical, or other interconnection).

As used herein, a compound is any molecular structure. Compounds can be described by their source, such as a living thing, such as a plant or animal, naturally occurring or manufactured, industrial, pollutant, food, and so on. Compounds also can be described by their typical activity with respect to other compounds, such as binding, agonist, antagonist, increasing response, decreasing response, partial agonist, partial antagonist, inverse agonist/antagonist, transcription modulation, phosphorylation, sequestration, catalyst, and so on. Compounds can be described by their compositional type, such as small molecule, macromolecule, large molecule, or polymer. Molecules may be organic or inorganic. Example organic molecules include but are not limited to lipids, alkaloids, nucleic acids, polypeptides, and so on. Example polymers include but are not limited to proteins, peptides, nucleic acids (e.g., RNA, DNA, or fragments thereof), glycan, or any combinations of the above.

Properties of items can include mechanical, optical, electrical, magnetic, electrooptical, electromagnetic, chemical, biological, or other properties (e.g., liquid, gas, solid, or other state) or any combination of these. To be useful in a machine learning context, the property of an item is quantifiable. The property can be any quantifiable information about the item which can be measured. For example, a property of an item can be quantified through laboratory experiments, field measurements, or other techniques. For example, a property of a compound can be a measured concentration of a response, such as a protein, in a sample in response to presence of a measured amount of the researched compound. Information that quantifies a property can be a value in a continuous range, in a piece-wise continuous range, or in a discrete range. The property can be measured with any sensor appropriate to the nature of the property.

Non-limiting examples of properties of a compound include, but are not limited to physical properties, reactivity, bioactivity (i.e., a quantifiable biological response of a living thing when the compound is present in or on the living thing), or biological properties. Example physical properties include molecular weight, protonation state, salt state, melting point, crystal structure, boiling point, density, length, volume, pH, and so on. Examples of reactivity include side chains (e.g., OH, COOH, NH₂, etc.), a number of bonds, a number of rotatable bonds, and so on. Examples of biological properties include the source of the compound (e.g., plant, animal, fungus, etc.), metabolism, and so on. A biological property can characterize a biological response as positive (i.e., healthy), negative (i.e., unhealthy), or neutral, or a combination of responses, such as a positive response such as reduction of a symptom and a simultaneous negative response such as a side effect. The information that quantifies a property can be a measurement of a characteristic of a reaction. This information can represent, for example, a concentration of a protein, a concentration of another item related to an amount of a protein, a concentration of RNA expression data, a readout from a sensor, such as luminescence, fluorescence, or radiation, or any other characteristic of the reaction that can be measured.

Data representing an item typically includes, or is processed to provide, a set of values for a set of features which distinguish the item from other items. Such features are used in machine learning as inputs to train a computational model or to use a trained computational model. The features may be a part of the data representing the item or may be derived from data representing the item.

For example, for a compound, the data representing a compound typically includes data defining the molecular structure of the compound. Data defining molecular structure of a compound can include any one or more of data representing: a molecular formula for the compound, a name for the compound, any isomers of the compound, a two-dimensional chemical structure of the compound, a SMILE string, three dimensional conformations of the molecule of the compound, any chemical property descriptors such as those calculated commonly with the RDKit software package, molecular properties, such as crystal structure, molecular weight, solubility. Such data, or any data resulting from transformation of such information, can be used as features which can be input to a machine learning model. As an example, features representing a compound can include a mapping onto a protein-protein interaction graph based on known compound-protein interactions, which is an ‘impact’-based featurization.

In some applications, the property of an item includes a value that depends on a quantity of the item. For example, the response elicited by a drug may depend on a dose of that drug. This value typically is known, or desired to be known, over a range of possible quantities of the item. For some quantities, a corresponding value may be unknown, partially known, or substantially known. Ultimately, such a property is actually a set of values, where each value corresponds to a respective quantity of the item.

A value that depends on a quantity of an item can be represented as a curve over a domain of possible quantities of that item. In biology, such a curve is typically called a dose-response curve. In practice, laboratory experiments or physical measurements result in respective values for specific quantities of the item. In some implementations, such data may be represented by and stored in an array data structure. Such an array of values for corresponding quantities may be incomplete for any given researched item. In some implementations, the data representing the item quantity dependent values can be a set of values for a set of parameters of a model that describes the curve.

Thus, a property of a researched item which is known can be a set of values for a set of quantities of that researched item, whether represented by a set of discrete values or by parameters of a model describing the curve. Similarly, a property of a predicted candidate item can be a predicted set of values for a set of quantities of that researched item, whether represented by a set of discrete values or by parameters of a model describing the curve. For some predicted candidate items, the property can include a label which indicates whether the item is “active” or “inactive”. For inactive items, a representation of any curve or set of values can be omitted and a label can be used instead.

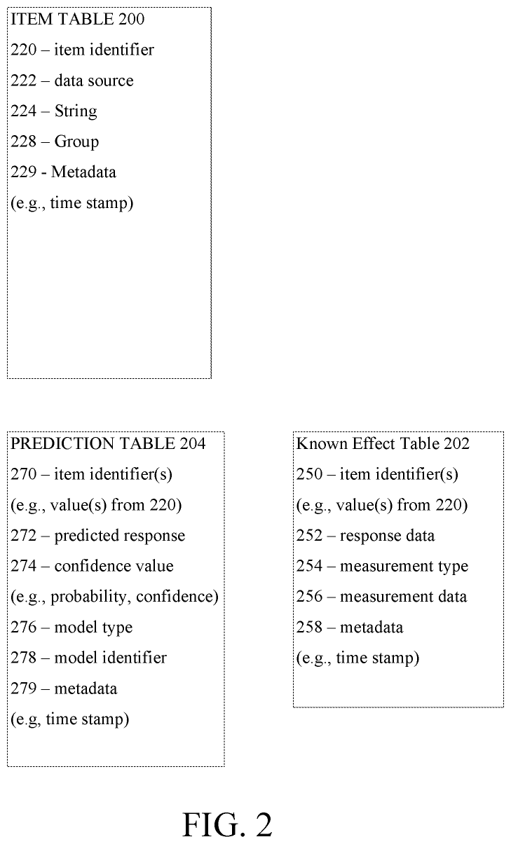

Having now described items, properties of items, item quantity dependent values, and the relationship among researched items, potential candidate items, and predicted candidate items, and an architecture of a computer system in FIG. 1, illustrative data structures for an example implementation of such a computer system for the purposes of researching item quantity dependent values, such as dose-response curves for compounds, will now be described in connection with FIG. 2.

Such data structures can be implemented, for example, using one or more tables in a relational database, or using one or more data objects in an object-oriented database, or using one or more documents in a NoSQL database, or by using data structures allocated in memory for an executing computer program, or by using any data structures implemented through other programming techniques. The use of database tables in the following examples is merely illustrative.

As shown in FIG. 2, an item table 200 can be used to represent all items, whether researched items, potential candidate items, or predicted candidate items.

Another table, herein called a known effect table 202, includes data representing information characterizing properties, such known item quantity dependent values for items. Thus, if an item has known item quantity dependent data for a property, making it a researched item with respect to that property, then the item has an entry in the known effect table 202 which includes information characterizing that property.

Another table, herein called a prediction table 204, includes data representing predicted properties for items. Thus, if an item has been predicted to have certain item quantity dependent values for a property, making it a predicted candidate item with respect to that property, then the item has an entry in this table that includes information, herein called prediction data, describing that prediction.

Additional details of an example implementation of the tables in FIG. 2 will now be described. It should be understood that this example is merely illustrative, as a variety of information can be stored in the database in diverse ways.

In this example, item table 200 includes data representing each item. For each item, information such as an identifier 220, can be stored. This identifier can be used as a primary join key with other tables. A suitable identifier is any form of data that can uniquely identify an item and distinguish the item from among other items in the system. One or more of such identifiers can be stored, allowing processing of the table in diverse ways.

The data representing an item can include an indicator 222 of a source, such as a database or laboratory experiment, from which information about the item has been obtained. The data representing the item can include, for example, a string 224 of characters providing a name of or a description of the item. One or more of such strings can be stored. The string can directly or indirectly provide information about the item. For example, the string may be a reference to a data file defining information about the item.

The data representing an item can include group information 228. Items can be placed into groups, such as by categories, classes, or ontologies. A plurality of distinct groups can be defined. An item can be placed into one or more groups. Grouping of items enables other advantageous operations to be performed in the context of training and using computational models. For example, when specifying train/validate/test splits, placing members of the same group into the same split may reduce overestimating generalization, because predictions for members of the same group may be expected to be similar.

Other metadata 229 about the data representing the item can be stored. For example, a time stamp can be stored indicating when the entry for the item was created, the last time the data representing the item was modified or accessed. A variety of other metadata can be stored. For example, metadata about provenance of data stored in the system can be included, in addition to its source 222.

In the example shown in FIG. 2, known effect table 202 includes data representing known item quantity dependent values for researched items. In known effect table 202, a row for an item includes an item identifier field 250 which stores an identifier, e.g., item identifier 220, of the item.

The response data field 252 stores data related to the item quantity dependent values for a property that are known for this item. For example, this field can store an identifier of a record that stores experimental data, or an array of that experimental data, or parameters of a curve representing known values for experimental data. In some implementations, an array of measurements indexed by item quantity can be used, representing a mapping of different quantities of an item to its measured response. In some implementations, such an array may be, and typically is, incomplete. In some implementations, there may be multiple entries for each item, with each entry storing a different response value.

The known effect table 202 can further associate this information with data indicating a type 254 of measurement, assay, or experiment used, and any other data 256 about that measurement, assay, or experiment. Other metadata 258 about the known response data also can be stored. For example, time stamps can be stored indicating when the data was created or the last time this data was modified or accessed.

In the example shown in FIG. 2, prediction table 204 includes data representing predicted item quantity dependent values for other items, herein called predicted candidate items. Data in the prediction table 204 are populated as a result of training a computational model using data about selected researched items and a selected type of property, and then applying the trained model to data about selected potential candidate items.

In prediction table 204, in a row for an item, an item identifier field 270 stores an identifier for the item. The predicted response field 272 stores data related to the item quantity dependent values for a property that have been predicted for this item. For example, this field can store an identifier of a record that stores predicted data, or an array of predicted data, or predicted parameters of a curve that represent such values. In some implementations, the prediction response can be the set of values for a set of parameters describing a Hill model representing the predicted response curve for different quantities of the item and their predicted response, such as a dose-response curve.

Prediction table 204 can further associate this information with data which helps to interpret the predicted response. For example, a confidence value 274, such as a probability or confidence score, or a class 276 indicating a type of machine learning model that generated the prediction, can be stored. Different types of machine learning models generate different kinds of confidence values 274, such as a probability, a confidence, a classification, or other output or combination of outputs. An identifier 278 of the computational model that resulted in this prediction also can be stored. Other metadata 279 about the prediction can be stored. For example, a time stamp can be stored indicating when the data was created or the last time this data was modified or accessed.

The training and use of a computational model which accesses and uses the data in the item table 200 and known effect table 202 to generate the data in prediction table 204, will now be described in more detail by way of an example, illustrative implementation.

As explained above, using a computational model and a training set of data, e.g., data about researched items, a model training system trains the computational model using a machine learning algorithm, features derived from the training set, and supervisory information available for or derived from the training set. The trained computational model is then applied to a target data set, e.g., a set of potential candidate compounds, to make predictions about the target data set.

A computational model used in a machine learning application typically computes a function of a set of input features, which may be a linear or non-linear function, to produce an output. The function typically is defined by mathematical operations applied to a combination of a set of parameters and the set of input features. Machine learning involves adjusting the set of parameters to minimize errors between the function as applied to a set of input features for a set of training samples and known outputs (supervisory information) for that set of training samples. The output of the computational model typically is a form of classification or prediction.

Such computational models are known by a variety of names, including, but not limited to, classifiers, decision trees, random forests, classification and regression trees, clustering algorithms, predictive models, neural networks, genetic algorithms, deep learning algorithms, convolutional neural networks, artificial intelligence systems, machine learning algorithms, Bayesian models, expert rules, support vector machines, conditional random fields, logistic regression, maximum entropy, among others.

The output of a computational model is a prediction value indicative of whether, or to what extent, an item has, if any, a selected property. This prediction can be in the form of, for example, a probability between zero and one, or a binary output, or a score (which may be compared to one or more thresholds), or other format. As a specific example, for a single item and corresponding property, values for parameters describing a one-dimensional Hill model, or other relevant model, may be known or predicted. The output can be accompanied by additional information indicating, for example, a level of confidence in the prediction. The output typically depends on the kind of computational model used.

A training set generally comprises a set of samples for which respective information about each sample is known, i.e., a set of researched items. Data called “features” are derived from information available about the samples the training set. These features are used as inputs to a computational model. The known information for the samples, typically called “labels,” i.e., the information characterizing the known properties of the researched items, provides the supervisory information for training. The supervisory information typically corresponds to the desired outputs of a computational model. A computational model has parameters that are adjusted by the training algorithm so that the outputs of the computational model, in response to the features for the samples in the training set, correspond to the supervisory information for those samples. Most training algorithms divide the training set into training data and validation data. Given a trained computational model, the trained computational model can be applied to features derived from the data for potential candidate items. The trained computational model provides an output indicative of a prediction about the potential candidate item.

Using the example data structures shown in FIG. 2, data for a training set can be specified by a query (or an identifier for such a query) on the item table 200 joined with entries from the known effect table 202 that contain one or more selected values as the effect identifier. Similarly, data for the potential candidate items can be specified by a query (or an identifier for such a query) on the item table 200 for items which are not in the training set, and which satisfy any other criteria desired.

Given a training set, the model training system 105 (FIG. 1) trains a computational model. Generally, a training algorithm applies, as inputs to the computational model, features derived from the data representing the researched items in the training set. Outputs from the computational model are obtained and compared to the supervisory information corresponding to those inputs. Parameters of the computational model are modified so as to reduce the errors between the outputs obtained and the supervisory information. The training algorithm involves iterating these steps of applying, comparing, and modifying until such errors are sufficiently reduced.

After the computation model is trained, the trained model execution system 107 applies the trained computational model 106 to the data representing some potential candidate items. The trained computational model thus generates and stores a result set. The result set includes a set of predicted candidate items (110 in FIG. 1) identified from among the plurality of potential candidate items as likely to have a property. Such information can be stored, for example, in a data structure such as shown as a prediction table 204 in FIG. 2.

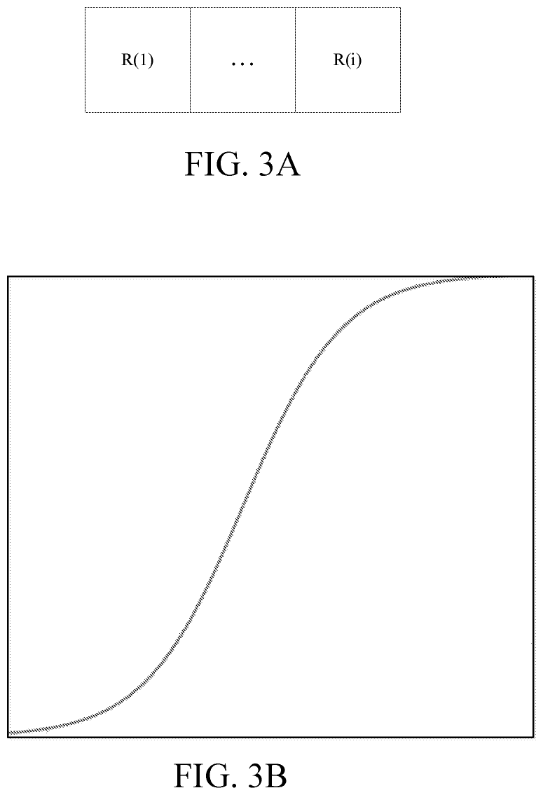

Turning now to FIGS. 3A and 3B, an item quantity dependent value for an item will now be explained.

FIG. 3A illustrates an array of data representing different values of an item that correspond to different quantities of an item. Such data may represent, for example, different responses, such as protein expression in a cell, in response to different quantities of an item, such as doses of a drug. In this example, different quantities of the item are shown on an axis i, and the corresponding values are illustrated R(1), . . . R(i). The set of values is represented by an array R, with index i, a positive integer, with each value Rᵢ being indicative of the value corresponding to the quantity i of the item.

FIG. 3B illustrates an example curve representing a set of item quantity dependent values. In this example, the curve is sigmoidal. There are many possible kinds of curves which can represent item quantity dependent values. However, most dose-response curves in biochemistry are sigmoidal. In any particular application, a curve that can be fit to experimental measurement typically can be expressed mathematically as a parametric function which is continuous. In the example in FIG. 3B, it is assumed that a mathematical model of a sigmoidal curve has a minimum value, a maximum value, a midpoint (typically called the “C” parameter of a Hill curve), and a slope at its midpoint.

A computational model is trained using a training set of researched items, including data representing known sets of values for items and other data representing those items. Input features to the computational model include data representing the item, and an output includes a set of values which depend on different quantities of the item. In training, this output can be compared to supervisory data representing the known sets of values for different quantities for the items in the training set. The result of this comparison can be used to update parameters of the computational model.

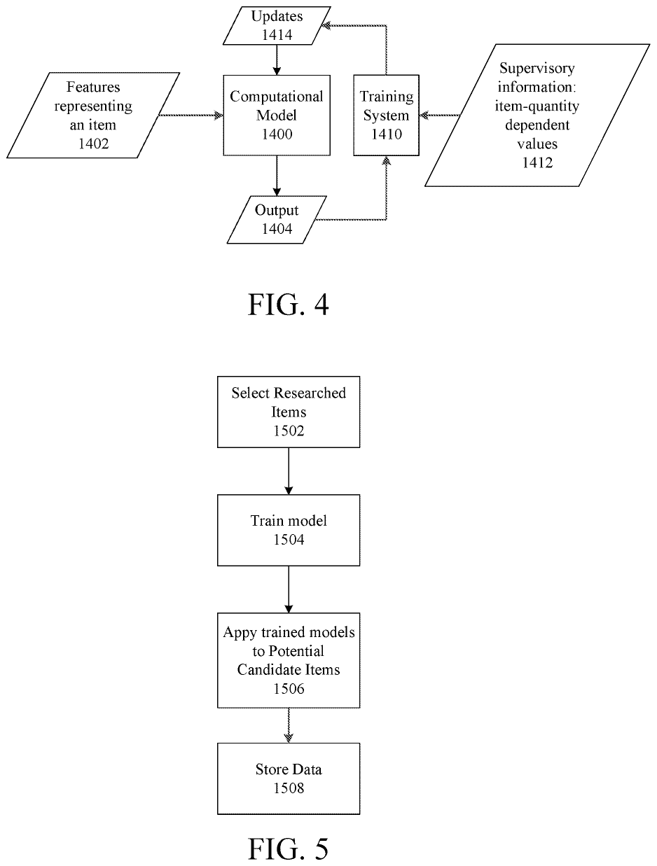

FIG. 4 is a data flow diagram of an example implementation of a computer system that incorporates a computational model for predicting item quantity dependent values.

In this example, a single computational model 1400 is illustrated, but any number of two through any positive integer N models can be used, and ensembled together. In this example, the computational model 1400 receives a set 1402 of input features for an item (whether a researched item during training, or a potential candidate item during application of the trained model). To generate input features, given a set of items, the data representing those items are processed into a set of values for a set of features. The features used for any particular type of item are selected to capture similarity or differences or both among items of that type. The computational model 1400 provides an output 1404 from which a representation of the item quantity dependent values for the item can be derived. As such, the computational model performs a weighted mapping of the input features into a domain of values that represents a dose response curve, In many biological and chemical processes, this dose response curve is represented by a Hill curve. Parameters of a model that define the response curve, or a corresponding array of values sampling that curve, are predicted by the computational model. In some implementations, the parameters of the curve can be processed into an array of values to allow for supervision of training using an array of values, and vice versa.

A training system 1410 trains the computational model 1400 using a training set including data representing known properties of researched items and other data representing those researched items. Input features include data representing the researched item. The output 1404 includes values from which the model's predicted representation of the item quantity dependent values for the researched item can be derived. For example, the output can be an array of values or parameters of a curve representing such values.

In training, the output 1404 is compared to supervisory information representing the known information about the item quantity dependent values for that researched item. For example, the supervisory information can be an array of values or parameters of a curve representing such values. The result of this comparison can be used to compute an update 1414 to parameters of the computational model 1400, as described in more detail below.

FIG. 5 is a flowchart describing an example operation for training and using a computational model. Training starts with specifying the training set to be used to train a computational model, specifics of the computational model, and a set of potential candidate compounds to which the trained computational model will be applied. At 1502, data representing the researched items are accessed. Then, at 1504, the training system trains the computational model using the training set using the data representing the researched items and their known item quantity dependent values. At 1506, a trained model is applied to a set of potential candidate items (PCI). The trained model outputs prediction information for each potential candidate item. The prediction information can be stored (1508) in the database (e.g., data structure 214 in FIG. 2). In some implementations, if the prediction information indicates that the potential candidate item does not have any useful data for the property, the prediction information for that item can be dropped and not stored.

In such a system, the computational model can be any model that takes, as an input, a set of features representing an item, and provides as an output an array of values representing a set of item quantity dependent values for a property for the item, or parameters of a curve representing such values. For training, parameters of the computational model can be updated based on errors between the output values and the supervisory information. An example model of such a type is described in more detail below in connection with an example relating to predicting dose-response curves describing bioactivity in response to compounds.

In the following two examples, which relate to combinations of compounds and bioactivity that occurs in response to the compounds together, two examples of different features are used.

In the first example, data representing compounds is processed into features based on molecular structure information. A technique such as described in Yang, Kevin, et al., “Analyzing Learned Molecular Representations for Property Prediction”, in J. Chem. Inf. Model., 2019, 59, 3370-3388, can be used for this mapping of compound chemical structures into features.

In the second example, data representing compounds is processed into features based on a protein-protein interaction framework where compounds are mapped onto a protein network based on known or predicted compound-protein interactions. A graph neural network is trained with a training set to produce a set of features. At inference/prediction time, a set of potential candidate items is input to the graph neural network that generates a set of values for a set of input features for the items. For example, a technique such as described in Gonzalez, Guadalupe, et al., “Predicting anticancer hyperfoods with graph convolutional networks”, in Human Genomics, 2021, 15:33 can be used for this kind of mapping of compound information into features.

In either case, or with other featurizations, the features are input into a computational model that maps the input features to a set of values for a set of respective quantities which represent the dose-response curve of the compound. In many applications, the dose-response curve is a one-dimensional variable slope sigmoidal curve, which typically can be represented by a Hill curve, which can be specified by a set of parameters.

An example of parameters that describe a response curve in the form of a one-dimensional (1D) Hill curve are the following in Table I:

TABLE I

| | |
|---|---|
| C | The concentration of drug required to achieve 50% of the maximal |
| | effect (i.e., EC₅₀). |
| h | Hill coefficient for the dose-response curve of the compound. |
| E₀ | The basal effect (where the dose is zero). |
| E₁ | Maximal efficacy of the compound. |

In one implementation, the computational model outputs a set of values for these parameters based on the features representing items. This set of values for the set of parameters predicted by the computational model can be converted into an array of values representing the dose response curve of the compound.

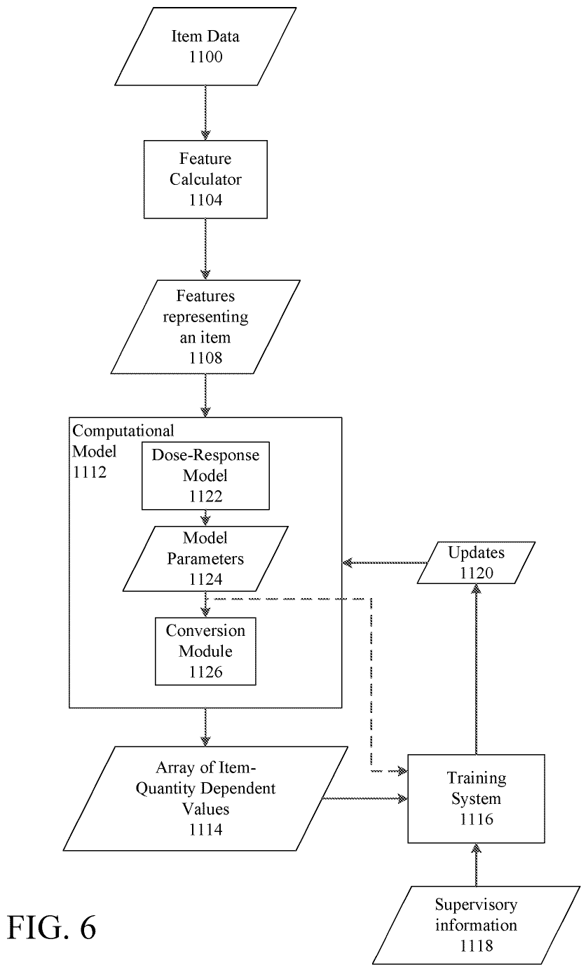

For example, a deep learning predictive machine learning model can be used, and can be configured so that the predictive output of the model takes the form of a predicted dose-response curve defined by such parameters. For example, a fully-connected network can have inputs that receive the features output by the graph neural network (GNN) described above, and the output of the model is configured to predict a Hill curve with regularized shape parameter values. In the example shown in FIG. 6, described in more detail below, these parameters are then processed to generate an array of values. These parameters thus serve as an intermediate parameterization, establishing a latent parameterized space of the machine learning model. This machine learning model can be supervised using the parameter values (i.e., supervised in the latent space) or supervised directly on the output array of values (i.e., supervised in the output array space).

FIG. 6 illustrates this combination of transformations of the data representing items to the data representing item quantity dependent values. Data representing an item 600 is processed by a feature calculator 604 to generate a set of values 608 for a set of features representing the item. These features are the inputs to the computational model 612.

The computational model 612 includes a dose-response model 622 which, in response to the features representing the item, outputs a set of values for the parameters 624 representing a dose-response curve representing the dose response of the item. Thus, the dose-response model itself includes parameters (not shown) which affect how the model generates the output values for the parameters 624. The set of values for the parameters output by the dose-response model are inputs to a conversion module 626. The conversion module computes an array of values representing a set of item quantity dependent values for different quantities of the item, based on a function of the parameters 624.

Instead of predicting the dose response for an item at certain quantities, this computational model predicts parameters that describe the full response curve. Predicting the full response curve provides a better understanding of the effect of the compound throughout a range of possible quantities. In the context of drug response for combinations of drugs, this allows a therapeutic window to be more easily identified, and to better understand drug interaction effects. These parameters can be converted into specific values for specific quantities of the item.

During training, the output 614 of the computational model 612 is provided to a training system 616, which also receives the supervisory information 618 for each item in a training set. In some implementations, the set of values for the parameters output by the dose response model 622 can be used as an input to training. Based on the error between the predicted values 614 and the supervisory information 618, updates 620 to the parameters of the computational model are generated and applied to the computational model.

With the two stages of modeling, the system first infers biophysical model parameters, then uses those parameters to generate observables, i.e., the quantified response for different quantities of the item. Predicting observables from a parametric model imposes biological prior information on the predictions (regularization of functional form) while also resolving degeneracy in the inference of the latent model parameters. That is, the model parameters may be poorly identified by ground truth observational data of dose response, which means that for a given parameter, there are many values that can lead to approximately the same dose response curve. Thus, there are benefits to supervising with an array of quantified responses as the ground truth data, which is definitely measurable, and not with inferred dose response curve parameters. In particular, the resulting model can infer a range of possible latent parameter values for a dose response curve corresponding to the quantified observational data.

Using such a model to predict item quantity dependent values has a particular complication. Within a training set, it is generally considered good practice to have both positive and negative examples of items with respect to a property. In the example of dose-response curves of compounds and related bioactivity, it is desirable to have, on the one hand, some researched items for which there is no significant bioactivity, and, on the other hand, some researched items for which there is known significant bioactivity and for which the dose-dependence of that bioactivity also is known at least in part. However, building a model that predicts both whether a compound has significant bioactivity and the item quantity-dependency of that bioactivity can challenge conventional machine learning techniques, because such predictions involve both classification problems and regression problems.

To address the problem that the model is performing both classification and regression, a dynamic loss function is used. In particular, the loss function is a dynamically weighted combination of two loss function components. The first loss function component optimizes the model for classification; the second loss function component optimizes the model for regression, or curve fitting. In one implementation, the loss function component is initially heavily weighted in favor of classification. This initial weighting reduces the likelihood that the model attempts to fit the data for an item to a curve where that data instead indicates that the item does not have quantity dependent values. For example, if a compound does not have a certain bioactivity, then the model should not attempt to map that compound's data to a dose-response curve for that bioactivity. As training epochs progress, the weighting towards the regression-based loss function increases.

An example implementation of such a dynamic loss function includes a function that implements, at training epoch i, a combination, using a weight (Wᵢ), of a first classification-based loss function (Aᵢ) and a second regression-based loss function (Bᵢ), such as Wᵢ(Aᵢ)+(1−Wᵢ)(Bᵢ). As an example, such a function can take the following form:

$${W}_{epoch} = min ( 1 , 1 / ( dynamic\_weight * epoch )$$

$${Loss}_{epoch} = {W}_{epoch} ( {Loss ( MSE )}_{epoch} ) + ( 1 - {W}_{epoch} ) ( {Loss ( CE )}_{epoch} )$$

where dynamic_weight is the number of epochs to have contribution from the classification function, and W_{epoch} is the weight for the regression model and is dependent on the current epoch of the model, and Loss (MSE) is the loss computed based on minimizing the square error for the current epoch, and Loss (CE) is the loss computed for the current epoch based on minimizing the cross entropy between the predicted activity classification label and the experimental values. Some typical initial values of the dynamic_weight parameter are 0 (no contribution from classification), 30 (classification will contribute for 30 epochs) and 1000 (or the specific maximum number of training epochs).

In some implementations, it is helpful to incorporate uncertainty modeling into the computational model. Uncertainty modeling relates to discounting predicted activity of a primary model by, for example, predictions of a secondary model or through specialized post-processing of the predictions of the primary model. The secondary model can be any uncertainty model that can assess the reliability of the primary model. As an example, an uncertainty model can assess differential reliability of a deep neural network (DNN). As another example, an uncertainty model can be an analytical approximation of the uncertainty of the primary model.

An uncertainty model can be in itself a computational model that outputs its own prediction value. The input features for the uncertainty model can be derived in several ways, such as one or more of the following techniques. For example, the input features can be generated using various embedding techniques, such as autoencoders or other transforms, based on the data about the items processed by the primary model. The input features may include the output predictions of the primary model. The input features can include all or a subset of the input features of the primary model. Herein the prediction value output by the uncertainty model is called the “uncertainty value” to distinguish it from the prediction value output by the primary model of which reliability is being assessed. In some implementations it is desirable to assess the suitability of the uncertainty model.

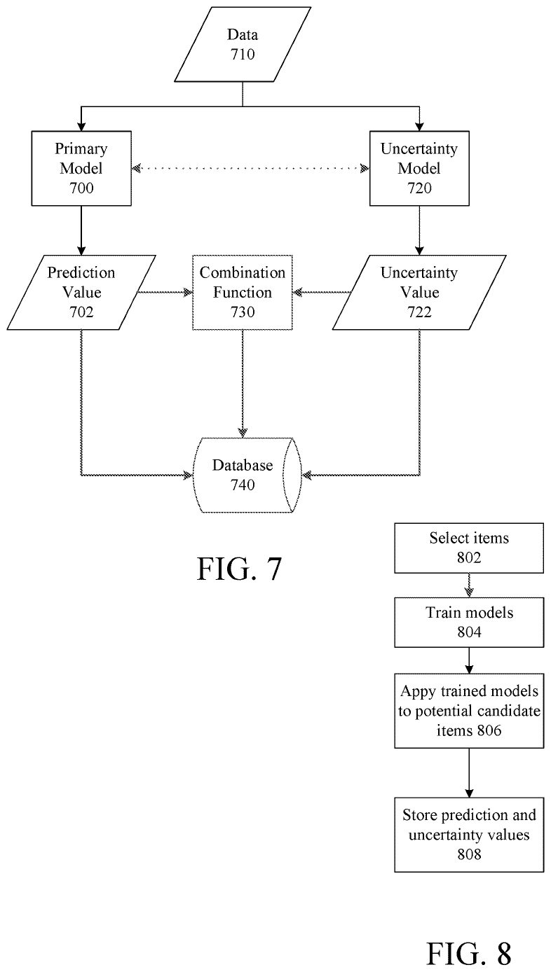

Thus, as shown in FIG. 7, in an example implementation of a computational model incorporating uncertainty modeling, a primary model 700 is the computation model (e.g., model 1400 in FIGS. 4 and 6) that generates the primary prediction values 702 for an item, using data 710. The illustration in FIG. 7 assumes the primary model and uncertainty model already have been trained. For each prediction, the uncertainty model 720 generates an uncertainty value 722. The uncertainty value also can be stored in the database 740 of results (e.g., table 204 in FIG. 2) along with the prediction value 702. A combination function 730 implements one or more functions that combine the prediction value and the uncertainty value. The result of this combination function also can be stored in the database 740 or computed in real time when requested. One or more combination functions can be used, and storage of the prediction value and uncertainty value in the database allows different combination functions to be applied at various times and for different purposes.

Examples of such an uncertainty model include, but are not limited to, the following. One or more uncertainty models, including models of diverse types, can be used in combination.

As one example, a deep ensemble-based model measures variance across an ensemble of primary models, each of which is trained with different random seeds and data subsets. An example of a model is described in “Simple and Scalable Predictive Uncertainty Estimation,” by Ralaji Lakshminarayanan, et al., available at arXiv: 1612.01474v3. Another example of a model is described in “Evaluating Scalable Uncertainty Estimation Methods for DNN-based Molecular Property Prediction,” by Gabriele Scalia et al., and available at arXiv: 1910.03127.

In some implementations, a function based on a sum of the uncertainty value and the prediction value can be computed, effectively representing an upper confidence bound. In some implementations, a function based on subtracting the uncertainty value from the prediction value can be computed, effectively representing a lower confidence bound. Weights can be applied to uncertainty values in such functions. In ensembles of primary models, multiple independent uncertainty estimates also can be used in combination.

FIG. 8 is a flowchart describing operation of a computational model incorporating uncertainty modeling. This process is applied to a specified computational model, with a specified training set to be used to train the computational model, and a specified set of potential candidate items to which the trained computational model will be applied, as identified at 802. The specification of the computational model can include a specification of the primary model and the uncertainty model. There can be more than one uncertainty model. The combination function used to combine the prediction values and the uncertainty values also may be specified, or may be specified and applied at a later time.

At 804, the computational model and the uncertainty model are trained using a machine learning algorithm and the training set. This includes training the primary model using the specified training set and training the uncertainty model using the specified training set along with any auxiliary tasks, embeddings, autoencoders, or augmented data.

At 806, the trained models, both the primary model and the uncertainty model(s), are then applied to the set of potential candidate items. The trained primary model outputs a prediction value for each potential candidate item and the type of combined effect. The trained uncertainty model outputs an uncertainty value for each potential candidate item and type of combined effect. These values can be stored (808) in the database, e.g., in table 214. In some implementations, a combination of the prediction value and the uncertainty value can be used to determine whether an item should be identified as a predicted candidate item for which the values should be stored in the database.

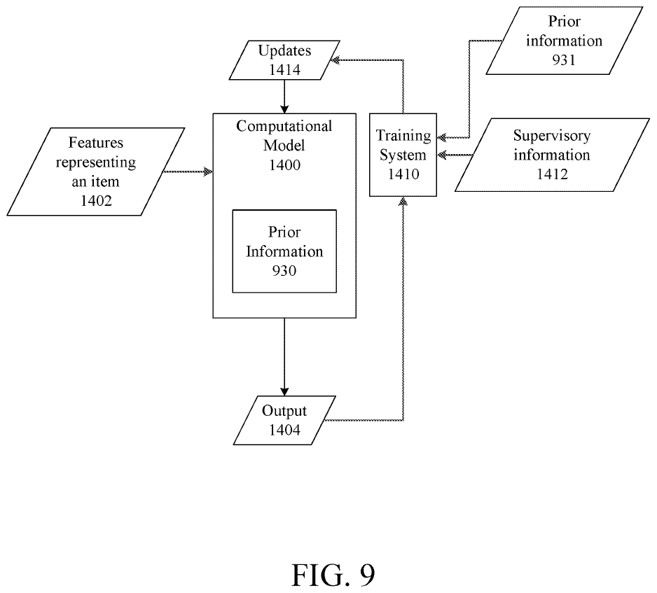

Turning now to FIG. 9, the potential problem of unrealistic predictions will now be addressed. In some implementations, prior information, such as data available for researched items, can be used to ensure that the computational model outputs predicted effects within a physically likely range. The prior information can be data representing a predetermined likely distribution of item quantity dependent values. The prior information can be data representing upper or lower bounds, or both, for the item quantity dependent values. Such prior information can be used during training or during inference.

In FIG. 9, the components of the machine learning system are similar to those found in FIGS. 4 and 6. In some implementations, the computational model 1400 can include prior information 930 as a form of limiter on the output 1404 of the model. For example, it may be known that the item quantity dependent values cannot be larger than some maximum value, or cannot be smaller than some minimum value, or both. A final stage of the output of the computational model can be to limit the output value to one or both of such values.

In some implementations, prior information 931 can be used by the training system 1410 to adjust the parameters of the computational model. In such implementations, the prior information acts as a form of supervisory information. The output 1404 can be compared to the prior information 931 to compute an error that the training algorithm can minimize.

For example, when fixed upper or lower limits are applied as constraints, they can be integrated through either parameter transformations or asymptotic penalties. The parameter transformations convert untransformed parameters, modeled in an unbounded real number space, to a bounded space (e.g., positive definite) using e.g., an asymptotically logarithmic transformation function.

In some cases, the prior information may be a distribution of likely values for one or more parameters output by the dose response model. This distributional prior information is integrated as a regularizing penalty function in the model's loss function during training. This allows the model fitting algorithm to explore any real valued number for the parameter, but penalizes samples according to their consistency with the prior distribution.

In some cases, the conversion module applies prior information about the functional form of the applicable dose response curve. The choice of conversion function can regularize and constrain the family of output models producible by the framework.

This approach integrates the Bayesian conception of domain-expert designed prior information on latent variables directly into a deep learning modeling framework that seeks to objectively optimize a multi-layer network of weights. This is possible because of the integration of the physically-motivated parameters output by the synergy model as an intermediate output of the deep learning model.

When using two or more computational models that predict item quantity dependent values, it is possible to combine the outputs of the models using a form of ensembling operation. For example, in some implementations the ensembling operation can compute an average of the arrays output by the models for a given item. In some implementations a form of Bayesian stacking can be used. In some implementations, the arrays output by the models can be weighted before ensembling, based on one or more pieces of information. For example, weighting can be based on similarity metrics between the reference items in the training set and the potential candidate items. As another example, weighting can be based on uncertainty metrics output from uncertainty models associated with the predictions.

For example, the Bayesian hierarchical stacking technique averages model instances in an ensemble where a different weight vector across model instances is generated for each sample. In particular, the weight for each model instance is predicted as a function of covariant parameters. For example, if the sample is a combination of compounds, the covariates can specify the chemical class or structural properties of each compound or the union thereof. The model is trained using samples with ground truth outcome labels and then can predict optimal model weights applicable to novel items.

Such machine learning models have several practical uses.

Generally, these techniques involve predicting the parameters of curve representing how a value is dependent on an item quantity, where the curve (i.e. the shape of a physical response or its mathematical functional form) is known based on domain knowledge to represent how that value typically depends on item quantity across a class of items. The resulting model is both data efficient, by allowing use of disparate data modalities, and regularizing, which allows small data sets to be used.

The shape of curve representing a physical response, or its mathematical function form, is often sigmoidal in the field of biochemistry, and often can be modeled using a Hill curve. Yet other parametric models of other curve shapes may be used. The shape of the curve to be selected can be obtained through domain expertise. The Hill curve is the most common type of equation to describe dose-response relationships for compounds and their bioactivity.

Some alternatives for representing biological dose response, or other types of biological responses and time series, include, but are not limited to, an Adair-Klotz equation, a generalized logistic function (also called a Richard's curve), a Gompertz curve or Gompertz function, or a Von Bertalanffy growth function (VGBF) (also called a Von Bertalanffy curve). The Gompertz and VGBF curves are typically used in the field of ecology to represent growth over time.

As further examples of other applications, parametric curves representing time series can represent such properties as growth of an organism, growth of tumors or bacterial cells, disease spread, consumer adoption of innovations, market impacts, and population growth.

As another example, the latent parameter model can be a form of constitutive model. Such models typically are used in materials science and mechanical engineering to analyze properties of objects, such as stress, strain, elasticity, and other properties. In such applications, a constitutive model represents each element of an object by a constitutive equation describing the physical phenomenon associated with the behavior of the element. The behavior of the object is modeled by a combination of the equations that describe each element. Such models can represent both linear systems and non-linear systems.

This technique can be used to combine single dose high throughput screening data and confirmatory dose response data into a single data set, to then predict, across a class of compounds, including those with sparse data in the training set, dose response curves for novel, untested compounds in that class.

The latent parameterization approach can be extended to areas such as material sciences and formulation development where observables such as time, particle size, impurity and composition, temperature, and other physical or chemical factors can be described by a parameterized mathematical model. Another example application is predicting power or performance behavior of a system, such as for various computing, electrical, mechanical, and power generation systems. Another example application is in predicting performance of materials, such as alloys or other materials. For example, the doping of a material with a dopant at different concentrations may result in different electrical properties. Another example application relates to properties of pharmaceutical formulations, or other products of chemical manufacture, or other articles of manufacture. For example, for different settings for 1) a temperature or other condition during a step of formulation, 2) a particle size or other property of a component, or other quantity that can vary can result in a change in a property of the resulting manufacture.

By making such predictions, laboratory experiments can be performed to validate the predictions. For example, an assay can be performed with a candidate compound and a selected protein to characterize the interaction of the candidate compound and the selected protein.

The foregoing description provides example implementations of a computer system implementing these techniques. The various computers used in this computer system can be implemented using one or more general-purpose computers, such as client devices including mobile devices and client computers, one or more server computers, or one or more database computers, or combinations of any two or more of these, which can be programmed to implement the functionality such as described in the example implementations.

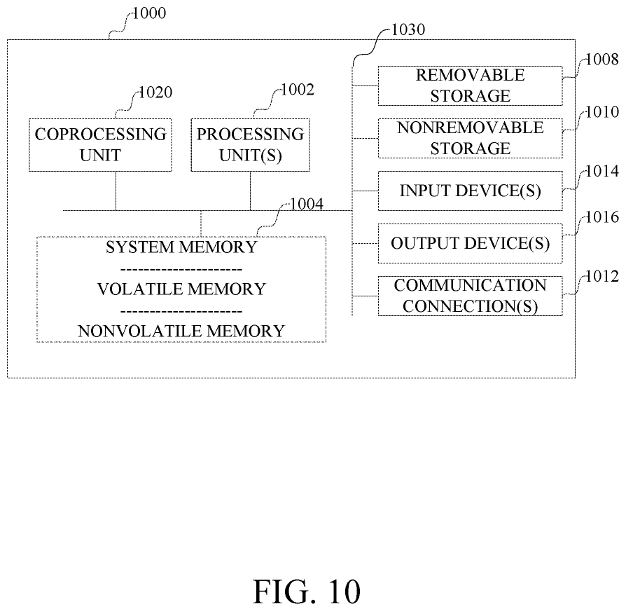

FIG. 10 is a block diagram of a general-purpose computer which processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, handheld computer, smart phone, media player, personal data assistant, audio and/or video recorder, or wearable computing device.

With reference to FIG. 10, an example computer 1000 comprises a processing system including at least one processing unit 1002 and a memory 1004. The computer can have multiple processing units 1002 and multiple devices implementing the memory 1004. A processing unit 1002 can include one or more processing cores (not shown) that operate independently of each other. Additional coprocessing units 1020, such as a graphics processing unit or field programmable gate array or other processor, also can be present in the computer. The memory 1004 may include volatile devices (such as dynamic random-access memory (DRAM) or other random-access memory device), and non-volatile devices (such as a read-only memory, flash memory, and the like) or some combination of the two, and optionally including any memory available in a processing device. Other memory, such as dedicated memory or registers, also can reside in a processing unit. Such a memory configures is delineated by the dashed line 1004 in FIG. 10. The computer 1000 may include additional storage (removable and/or non-removable) including, but not limited to, solid state devices, or magnetically recorded or optically recorded disks or tape. Such additional storage is illustrated in FIG. 10 by removable storage 1008 and non-removable storage 1010. The various components in FIG. 10 are generally interconnected by an interconnection mechanism, such as one or more buses 1030.

A computer storage medium is any medium in which data can be stored in and retrieved from addressable physical storage locations by the computer. Computer storage media includes volatile and nonvolatile memory devices, and removable and non-removable storage devices. Memory 1004, removable storage 1008 and non-removable storage 1010 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 1000 may also include communications connection(s) 1012 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 1012 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 1000 may have various input device(s) 1014 such as a various pointer (whether single pointer or multi-pointer) devices, such as a mouse, tablet and pen, touchpad and other touch-based input devices, stylus, image input devices, such as still and motion cameras, audio input devices, such as a microphone. The computer may have various output device(s) 1016 such as a display, speakers, printers, and so on, also may be included. These devices are well known in the art and need not be discussed at length here.

The various storage 1010, communication connections 1012, output devices 1016 and input devices 1014 can be integrated within a housing of the computer, or can be connected through various input/output interface devices on the computer, in which case the reference numbers 1010, 1012, 1014 and 1016 can indicate either the interface for connection to a device or the device itself as the case may be.

An operating system of the computer typically includes computer programs, commonly called drivers, which manage access to the various storage 1010, communication connections 1012, output devices 1016 and input devices 1014. Such access generally includes managing inputs from and outputs to these devices. In the case of communication connections, the operating system also may include one or more computer programs for implementing communication protocols used to communicate information between computers and devices through the communication connections 1012.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

In some implementations, computer program instructions for performing statistical processing can be implemented using the STAN statistical programming package, such as described in Stan Development Team, “Stan Modeling Language: User's Guide and Reference Manual”, accessible as a “manual.html” file at the mc-stan.org internet domain, and in Carpenter B, Gelman A, Hoffman M, Lee D, Goodrich B, Betancour M, Brubaker M A, Guo J, Li P, Ridell A, “Stan: A Probabilistic Programming Language.” Journal of Statistical Software, 76 (1), 1-32 (2017).

Such a system can be implemented in conjunction with a system as described in PCT Publication 2022/240751 or U.S. Provisional Patent Application Ser. No. 63/386,161, filed Jul. 12, 2022, entitled “PROCESSES, MACHINES, AND ARTICLES OF MANUFACTURE RELATED TO PREDICTING EFFECTS OF COMBINATIONS OF ITEMS”.

Each reference, e.g., non-patent publications, patents, and patent applications, cited herein is hereby expressly incorporated by reference herein in its entirety. In the event of conflict between subject matter herein and subject matter in such a reference, the subject matter herein controls.

It should be understood that the subject matter defined in the appended claims is not necessarily limited to the specific implementations described above. The specific implementations described above are disclosed as examples only.

What is claimed is:

1. A computer system for predicting an item quantity dependent property of items based on quantitative information obtained from measurements of the property of items, comprising:
a processing system comprising a processing device and computer storage storing computer program instructions that, when processed by the processing device, configures the processing system to:
train a computational model using data representing researched items,
wherein the data representing researched items includes, for each researched item, respective quantitative information describing item quantity dependent values corresponding to different quantities of the researched item,
input data representing a plurality of potential candidate items to the trained computational model such that the trained computational model outputs a result set,
wherein the result set includes respective predicted information, for each predicted candidate item from among the plurality of potential candidate items, describing item quantity dependent values for the predicted candidate item for a plurality of different quantities.

2. The computer system of claim 1, wherein the respective predicted information for each predicted candidate item comprises a respective set of values for a set of quantities of the item describing a sigmoidal curve.

3. The computer system of claim 1, wherein the respective quantitative information for each researched item comprises a respective set of values for a set of quantities of the item describing a sigmoidal curve.

4. The computer system of claim 1, wherein the respective quantitative information describing item quantity dependent values corresponding to different quantities of the researched item comprises sparse data for at least some of the researched items.

5. The computer system of claim 4, wherein the sparse data includes single dose high throughput screening data for a plurality of researched items.

6. The computer system of claim 5, wherein the respective quantitative information describing item quantity dependent values corresponding to different quantities of the researched item includes multiple dose data for at least some of the researched items.

7. The computer system of claim 1, wherein the computational model comprises a set of inputs receiving features representing items, a latent parameter model implementing a function mapping the features to outputs providing parameters representing a curve, wherein the curve represents how values for items depend on quantity of items in a class of items.

8. The computer system of claim 7, wherein the computational model further comprises a conversion module having inputs connected to receive the parameters representing the curve, a conversion model implementing a function mapping the parameters representing the curve to outputs providing data representing an array of item quantity dependent values corresponding to the curve.

9. The computer system of claim 8, wherein, to train the computational model, supervisory information includes the quantitative information describing item quantity dependent values corresponding to different quantities of the researched item.

10. The computer system of claim 9, wherein, to train the computational model, the processing system is configured to compare the supervisory information and the output of the conversion module to generate updates to parameters of the computational model according to a loss function.

11. The computer system of claim 10, wherein the parameters of the computational model include parameters of the latent parameter model.

12. The computer system of claim 10, wherein the parameters of the computational model include parameters of the conversion model.

13. The computer system of claim 10, wherein the loss function comprises a dynamic loss function.

14. The computer system of claim 13, wherein the dynamic loss function comprises a weighted combination of a first loss function and a second loss function.

15. The computer system of claim 14, wherein the first loss function comprises a classification-based loss function.

16. The computer system of claim 14, wherein the second loss function comprises a regression-based loss function.

17. The computer system of claim 14, wherein the dynamic loss function applies a weight that varies across training epochs during training.

18. The computer system of claim 1, wherein the items are compounds, and the item quantity dependent values are a dose-response curve.

19. The computer system of claim 18, wherein the dose-response curve represents bioactivity of the compound.
